(19) United States
(12) Patent Application Publication
(10) Pub. No.: US 2012/0178782 A1
(43) Pub. Date: Jul. 12, 2012

(54) COMPOSITIONS AND METHODS FOR TREATING PROLIFERATIVE DISEASES

(76) Inventor: Ernesto Jorge Podesta, Buenos Aires (AR)

(21) Appl. No.: 13/394,776

(22) PCT Filed: Sep. 10, 2010
(86) PCT No.: PCT/BR2010/000300

§ 371 Date: Mar. 7, 2012

(30) Foreign Application Priority Data
Sep. 10, 2009 (AR) P 2009 0103475

(51) Int. Cl. A61K 31/427; A61P 35/00; A61P 17/06; A61K 31/444

(52) U.S. Cl. 514/334; 514/369

(57) ABSTRACT

Synergistic pharmaceutical compositions and the methods for preventing and treating proliferative diseases such as cancer and psoriasis. The compositions comprise synergistic combinations of: (i) an acyl-CoA-synthetase enzyme inhibitor (AcsI4), (ii) a compound having an inhibiting effect on enzymes with cyclooxygenase activity (COX); and (iii) a compound selected from a 5-lypoxygenase enzyme (LOX-5) inhibitor and a leukotriene receptor antagonist.

### FIELD OF THE INVENTION

The present invention relates to synergic compositions and the methods for preventing and treating proliferative diseases such as cancer and psoriasis. More specifically, the present invention relates to synergic compositions and the methods for inhibiting the proliferation of cancer cells, particularly colon and breast cancer cells.

### PRIOR INVENTIONS

Among proliferative diseases, cancer is a disease in which abnormal cells multiply uncontrolled and can invade surrounding tissues. Cancer cells can also spread to other parts of the body through the blood stream and the lymphatic system. Breast cancer is the accelerated and uncontrolled proliferation of cells pertaining to distinct tissues of a mammary gland.

Cancer is not fully understood on a molecular level. It is known that exposing a cell to certain viruses, certain chemical compounds or radiation leads to DNA alterations that inactivate a suppressor gene or activate an oncogene. Suppressor genes are genes regulating growth, which, through mutation, can no longer control growth. Oncogenes is initially normal genes which, through mutation or altered context of expression, transform into transforming genes. The products of these genes produce improper cell growth or cell proliferation.

Cancer is currently treated mainly with a therapy selected among surgery, radiation and chemotherapy or with a combination of them. The effectiveness of these treatments is frequent limited, since cancer cells that spread from the location of the original tumor evade surgery and radiation. On the other hand, chemotherapy, which consists of administering cytostatic or cytotoxic medications, has the great limitation of insufficient specificity. The mechanism of action of these medications consist of causing now a cell alteration in nucleic acid synthesis, cell division or protein synthesis. The action of the different cytostatic drugs varies in accordance with the dosage given. Due to its lack of specificity, other normal cells and tissues of the body are affected, the side effects being related to these cells and tissues affected. The side effects depend on the chemotherapy agent, the most important being: alopecia, nausea, vomiting, diarrhea, constipation, anemia, immunosuppression, hemorrhage, cardiotoxicity, hepatotoxicity, nephrotoxicity, tumor lysis syndrome, etc.

There are new treatments focused on obtaining a better comprising of the series of events that take place in the transformation of a normal cell into an invasive cell with unregulated and disorganized proliferation. A specific treatment only directed at such cells would avoid damage to healthy tissues.

Recent studies have demonstrated that arachidonic acid metabolism has an important role in the development of cancer. Arachidonic acid is fatty acid of 20 carbon atoms that is a common constituent of the phospholipids in cell membranes. Through of a cell, arachidonic acid is released from the cell membrane, it later being able to be metabolized by means of cyclooxygenases, lipoxygenases and epoxygenases. Various lines of research indicate that the level of free arachidonic acid in cells is a critical sign of apoptosis, the cell mechanism for programmed cellular death (Yang Cao, A, Terrence Rearman, Guy A. Zimmerman, Thomases M. Mcintyre, Stephen M. Prescott, Intracellular unesterified arachidonic acid signals apoptosis, Proceedings of the National Academy of Sciences, 97: 11280-128:5).

An increase in the expression of lipoxygenases and its metabolites, leukotrienes, was detected in a variety of lines of cancer cells and in prostate, bladder, breast, lung, colon, pancreas, etc. tissues, and its overexpression was associated with the proliferation of tumor cells and resistance to apoptosis and angiogenesis. Published patent application WO 97/03667 describes the use of NSAIDs as cyclooxygenase inhibitors (COX), for the prevention of colon adenoma. Published application WO 96/41626 describes combinations of COX-2 and 5-lipoxygenase (LOX-5) enzyme inhibitors for treating inflammatory disorders, skin diseases like psoriasis, eczema and dermatitis, and in treating colorectal cancer. Published application WO 02/16575 describes a method for treating colon cancer, through administration of a compound that inhibits COX-2 and a compound that inhibits FACLA (Fatty Acid CoA, Ligase 4, also called acyl-CoA-synthetase). U.S. Pat. No. 6,756,399 B2 describes a method to treat a cancer derived from epithelial cells through administration of a combination of an arachidonic acid metabolism inhibitor and a PPAR ligand in an effective quantity to prevent or treat deal the growth of cancerous cells. With the exception of the dose of Indomethacin, the doses used for the combinations are doses effective per se to inhibit growth, it being indicated that the combinations used had an adjuvant effect.

The mechanism of action that characterizes or defines the effect of the NSAIDs (nonsteroid anti-inflammatory drugs) is the capacity to inhibit the activity of cyclooxygenase of the prostaglandin G/H synthetase enzyme and, therefore, to inhibit prostaglandin biosynthesis. NSAIDs prevents the formation of prostaglandin H2, the first step in the metabolism of arachidonic acid from a great cascade of formation of lipid signals that include the formation of prostaglandin D2, E2, F2α, I₂ and thromboxanes, the main prostanoid metabolite of platelets.

Two different forms of the prostaglandin G/H synthetase enzyme called cycloxygenase-1 (COX-1) and cycloxygenase-2 (COX-2) had been identified since the year 1991, COX-1 is constituently expressed in numerous tissues and has a fundamental role in platelet aggregation and in gastric cytoprotection. COX-2 constitutively expressed in the kidney and brain, but it can be induced in numerous tissues during inflammation, the production of wounds and in neoplasias.

NSAIDs varies in the capacity to inhibit COX-1 and COX-2, and this inhibition depends on the concentration and the tissue used. For example, aspirin is a selective inhibitor of the COX-1 enzyme in platelets in daily doses of 50-100 mg a day, but it can only inhibit COX-2 enzyme in plasma concentrations greater than 0.5 mM. However, other NSAIDs such as Ibuprofen, Sulindac and Indomethacin inhibit COX-1 and COX-2 in the same way. The new class of NSAIDs called coxibs by the World Health Organization selectively inhibits COX-2 and was developed to suppress the formation of prostaglandins during inflammation.

As previously presented, NSAIDs have also been used as anticancer drugs. The mechanism postulated to being anticancer agents is restoring the mechanism of apoptosis and, particularly for coxibs, inhibiting angiogenesis. However, the precise mechanism by which NSAIDs act is still controversial. Min Yao et al. “Effects of Nonselective Cyclooxygenase Inhibition with Low-Dose Ibuprofen on Tumor Growth, Angiogenesis, Metastasis, and Survival in a Mouse Model of Colorectal Cancer,” Clinical Research Cancer, Vol 11, 1618-1628, Feb. 15, 2005 1a., present a cell growth inhibition experiment employing Ibuprofen at a dose of at least 500 μM.

Leaving side enthusiasm to use NSAIDs as anticancer agents, particularly selective COX-2 inhibitors, there are still basic questions on the mechanism of action. This because the pharmacologic effect of NSAIDs is rather complicated due to the diverse functions of prostaglandins in different tissues. However, prostanoids can still have different effects according to the receptor with which they interact.

The family of thiazolidinediones (TZDs), including, among others, Rosiglitazone, Pioglitazone, Troglitazone, CS-011, AD-5075, BRL-49653, AY-31637, MCC-55, Darglitazone, Englitazone, Ciglitazone, 5-(4-)(2-(methyl-2-pyridylamine)etoxi)-phenol)methyl)-2,4-thiazolidinedionem, etc., are high affinity ligands for Peroxisome Proliferator Activated Receptors called PPARγ. This is transcription factor preferentially expressed in adipose tissue. These TZDs improve sensitivity to insulin or regulation of numerous aspects of adipose tissue function through transcriptional activation of genes sensitive to insulin, involved in glucose homeostasis, the fatty acid metabolism and the deposit of triglycerides in adipocytes. In addition, TZDs promote the differentiation of preadipocytes, mimicking certain genomic effects of insulin, modulating the expression of adiponectin, IL-6 type proinflammatory cytokines and TNFα. Through these beneficial effect, TZDs are used as an oral therapy for type II diabetes for reduction of resistance to insulin, helping in the control of glycemia. In the same way as adipocytes, it was stated that many human cancer cell lines have a high expression of PPARγ, and exposing these tumor cells in vitro to high doses (greater than or equal to 10 μM) of TZDs, specifically Troglitazone and Ciglitazone, leads to the arrest of the cellular cycle and to apoptosis, suggesting a supposed connection between the activated signal for PPARγ and the antitumor activity of TZDs. In addition, it was demonstrated in few clinical cases that Troglitazone had an anticancer efficacy in patients involved with liposarcoma. Until now, the identity of the target genes that contribute to the antiproliferative activity of PPARγ agonists is not known, and this remains without explanation. This is due in part to the complexity of genomic responses in PPAR activation and, despite the description of PPARγ genes associated with regulatory paths of cell growth, the maturity of epithelial cells of the colon in colorectal cancer, the functional role of these PPARγ target genes, which measure the antiproliferative activity of TZDs in cancer cells, is not clear. Moreover, many lines of evidence are against the fact that the antitumor effect is dependant on the activation of the PPARγ. For example, there is no correlation between the susceptibility of tumor cells to TZDs and the levels of expression of the PPARγ. It was suggested that the antiproliferative activity of TZDs could be associated with the inhibition of the Bcl-2/Bcl-xL function; with target protein degradation for proteasomes and with transcriptional repression of the androgen receptor through Sp1 degradation. There is evidence that suggest that Troglitazone and Ciglitazone reduce the intracellular association of Bcl-2 and Bcl-xL with Balk leading to apoptosis dependant on caspases. On the other hand, it is demonstrated that Rosiglitazone inhibits the activity of acyl-CoA-synthetase in smooth muscular cells of the human artery and in macrophages. It was described that inhibition of acyl-CoA-synthetase inhibits the proliferation of hepatocellular and colon cancer cell lines, Hong-Un Yu, et al. “Induction of G1 phase arrest and apoptosis in MDA-MB-231 breast cancer cells by Troglitazone, a synthetic peroxisome proliferator-activated receptor (PPARγ) ligand,” Cell Biology International 32 (2008) 906-912, present an experiment in cell growth inhibition by means of using Troglitazone in a dose of at least 25-50 μM, using Ciclitazone in a dose of at least between 50 and 75 μM and using Rosiglitazone in a dose of at least 75 μM.

5-lypoxygenase belongs to a family of enzymes that are expressed both in plants as well as in animals. Lipoxygenases enzymes, also called dioxygenases, catalyze the oxygenation of polyunsaturated fatty acids containing a 1,4-cis,cis-pentadiene motive to produce hydroperoxide derivatives. They require an atom of iron in the hemo per molecule that oscillates between iron 2+ in the inactive enzyme and iron 3+ in the active form during the catalysis cycle. Up until now, there are known three isoenzymes called lypoxygenase-5, lypoxygenase-12 and lypoxygenase-15, which correspond to the specific position of insertion of the oxygen group in the arachidonic acid that is its substratum. Knowledge of the biological role of lypoxygenase-12 and lypoxygenase-15 is limited. In contrast, it is known that lypoxygenase-5 participates in the formation of compounds that are powerful inflammatories.

The family of lypoxygenase-5 enzyme inhibitors is divided according to its mechanism of action in antioxidant compounds or redox inhibitors that intervene with the redox activation cycle of this enzyme, iron chelating agents and competitive inhibitors that compete with the substratum, arachidonic acid. There are indirect inhibitors that, upon joining with FLAP (5-Lypoxygenose-Activating Protein, 5-lypoxygenase activator protein), inhibits the activity of the enzyme.

Antioxidant substances are generally small aromatical molecules such as phenols, quinones and dihydroquinones. The prototype of this molecular class are the pyrazoline derivatives, Fenidione and BW-755C, Docebenone (AN-861) and Lonapalene (RS-43179). TMK-688 can also be mentioned (Linazolast), DuP-654, R-68151 and E-6080. In this same family are the compounds that act through redox reaction, such as BW-755C, ICI-207968 and A-53612. There is also a series of tetrahydro-1,2,4-triazien, R-85355 and R-68151. In the family of iron chelating inhibitors, such as derivates of N-hydroxyurea and hydroxamic acids, are Zileuton (A-64077) and Atreleuton (ABT-761). These compounds were evaluated in diseases like asthma. These compounds were introduced on the market in doses of 600 mg q.i.d. These compounds are used mainly as antiallergy drugs and in asthma therapy. They have demonstrated a variety of toxic effects mainly on the hepatic level. There are Zileuton derivatives like A78773. A79175 and A-76745 (Feuleuton).

Zileuton inhibits lypoxygenase-5 in the supernatant of 20,000×g of homogenates of leukemia basophil cells and in polymorphonuclear leukocytes of rat with an IC₅₀ of 0.5 and 0.3 μM, respectively; and in erythrocytes with an ID₅₀ of 0.9 μM. There is no information on inhibition of proliferation in cancer cells.

Among others types of compounds with the same inhibitory activity are known compounds derived from N-acetyl-hydroxylamine like BW-A4C and A63162, Various compounds were also discovered that form enantiospecific interactions with the enzyme, that is, methoxyalkylhyazoles and methoxytetrahydropyrone derivatives, like ZD-2138, ZM-230487 and hybrid molecules of natural products. ZD-2138 gave rise to compounds with better bioavailability such as L-697,198, L-708,780, L-739,010 and L-746,530. They even describe derivates of thiopyranilindols L-689.065, L-691.816 and L-699,333. Moreover, a series of compounds derived from di-hydrobenzofuran-N-hydroxyurea is also described like SB-210661 and SB-202235, heterocyclic compounds of hydroxyurea like CGS-26529 and CGS 23885. Finally, another type of compounds is described that interact with FLAP (5-Lypoxygenase-Activating Protein, 5-lypoxygenase activator protein) MK-866, as well as MK-0591 and Bay-X-1005.

Further, compounds are known that can inhibit lypoxygenase-5 and lypoxygenase-combined. These compounds are effective in allergy treatments in guinea pigs. They are derived from benzothiazoles E-3040 and the quinone derivative, CV-6505.

The inhibition of lipoxygenases, mainly the inhibition of lypoxygenase-5, was null for the inhibition of cell proliferation, Wei-Gang Tong et al, “The mechanisms of lipoxygenase inhibitor-induced apoptosis in human breast cells,” Biochemical and Biophysical Research Communications 296 (202) 942-948, shows an experiment in inhibition of breast cancer cell proliferation with the LOX-5 inhibitor, Rev-5901 (a derivative of arymethylphenyl ether) in a dose between 1.87 and 30 μM.

There are described two types of leukotriene receptors based on their biological activities. One of them refers to leukotriene B4 and is related to hydroxy acids and catalogued as BLT receptors. The other type of receptor is identified as cysteinyl-leukotriene. The different biological activity profile of these receptors gave rise to this classification. The cysteinyl-leukotrienes C4, D4, and E4 are important mediators involved in the pathogenesis of asthma. It is known that Zafirlukast is a selective and competitive antagonist of the receptor of leukotrienes C4 (LTC4), D4 (LTD4) and E4 (LTD4). Other antagonists are known for the cysteinyl-type receptor, such as Pranlukast Montelukast and Pobilukast are known, ICI-198615, MK-571, SR-260, RG-12525, WY-48252, SKF-106203, L-649923, FPL-55712. They all show different affinity for the receptor, Zafirlukast being the most powerful inhibitor and Ablukast, the least.

The use of combinations of agents like those previously described, for treating proliferative diseases, such as cancer and psoriasis, in quantities of per se inhibitory doses, suffers from significant disadvantages deriving from adverse side effects. In particular, conventional doses of NSAIDs such as Ibuprofen, Diclofenac, Naproxen, etc. involve increased risks of gastrointestinal disorders, from nausea, vomiting and gastritis to hemorrhagic ulcer. In addition, the use of certain COX inhibitors was associated with an increased risk of cardiovascular disease.

However, the use of acyl-CoA-synthetase enzyme inhibitors (AcsI4) such as thiazolidinediones in doses well below the minimum doses inhibiting cell proliferation is very important and desirable, given that, for example, in per se inhibitory doses of Troglitazone of 75 μM, toxic effects were described in cells of the microvascular endothelium in both mice as well as in humans (Satoko, Kakiuchi-Kiyota et al., Effects of the PPARγ agonist troglitazone on endothelial cells in viva and in vitro; Differences between lawman and mouse, Toxicology and Applied Pharmacology, 237 (2009) 83-90).

Consequently, the necessity persists to have a synergic combination of effective agents for preventing and treating proliferative diseases, in which the agents are administered in lesser doses than those conventionally used. Reducing the doses of the individual agents will diminish the incidence of adverse effects, thus improving the quality of life of the patient, even improving the fulfillment of the treatment by the patient and reducing the need to administer medications intended to relieve pain and the previously mentioned gastrointestinal disorders.

### SUMMARY OF THE INVENTION

The present invention foresees a pharmaceutical composition to inhibit cell proliferation which comprises a synergic combination of i) an acyl-CoA-synthetase enzyme inhibitor (AcsI4), ii) a compound having an inhibiting effect on enzymes with cyclooxygenase activity (COX), and iii) a compound selected from a 5-lypoxygenase enzyme (LOX-5) inhibitor and a leukotriene receptor antagonist.

The present invention also foresees a composition to inhibit the proliferation of cancer cells in which the composition comprises a synergic combination of: i) an acyl-CoA-synthetase enzyme inhibitor (AcsI4), ii) a compound having an inhibiting effect on enzymes with cyclooxygenase activity (COX), and iii) a compound selected from an inhibitor of the 5-lypoxygenase (LOX-5) enzyme and an antagonistic compound of leukotriene receptors.

In accordance with a particular embodiment, the cancer cells are breast cancer cells.

In accordance with another particular embodiment, the cancer cells are colon cancer cells.

In accordance with another embodiment, the present invention supplies a method for treating and/or preventing cancer in a patient who has need of such treatment, that comprises administering to the patient a synergic combination of: i) an acyl-CoA-synthetase enzyme inhibitor (AcsI4), ii) a compound having an inhibiting effect on enzymes with cyclooxygenase activity (COX), and iii) a compound selected from a 5-lypoxygenase enzyme (LOX-5) inhibitor and a leukotriene receptor antagonist.

In accordance with a particular embodiment, the cancer to be treated is breast cancer.

In accordance with a particular embodiment, the cancer to be treated is colon cancer.

In accordance with still another embodiment, the present invention foresees a method for treating and/or preventing psoriasis in a patient who has need of such treatment, that comprises administering to the patient a synergic combination of: i) an acyl-CoA-synthetase enzyme inhibitor (AcsI4), ii) a compound having an inhibiting effect on enzymes with cyclooxygenase activity (COX), and iii) a compound selected from a 5-lypoxygenase enzyme (LOX-5) inhibitor and a leukotriene receptor antagonist.

### BRIEF DESCRIPTION OF THE DRAWINGS

FIG. 1 shows the dose/inhibition response results of cell proliferation of a breast cancer cell line by using different inhibitors in distinct concentrations. FIG. 11A shows the proliferation percentage results of acyl-CoA-synthetase inhibitors; FIGS. 1D and 1C show the proliferation percentage results of COX inhibitors; FIGS. 1D and 1E show the proliferation percentage results of LOX inhibitors; and FIG. 1F shows the proliferation percentage results of a leukotriene receptor antagonist compound.

FIG. 2 shows the effect on cell proliferation of a breast cancer cell line measured by MTT assay, by combining pairs of inhibitors in concentrations that do not have any effect per se. FIG. 2A shows the dose/response results of COX and LOX inhibitors; FIG. 2B shows the dose/response results of LOX and acyl-CoA-synthetase inhibitors FIG. 2C shows the dose/response results of COX acyl-CoA-synthetase inhibitors; FIG. 2D shows the dose/response of the combination of COX inhibitors, acyl-CoA-synthetase inhibitors and LOX inhibitors.

FIG. 3 shows the effect on cell proliferation of a breast cancer cell line measured by MTT assay, by combining pairs of inhibitors used in the minimum concentrations that produce inhibiting effect.

FIGS. 4A and 4B show the inhibiting effect on cell proliferation of a breast cancer cell line, measured by MTT assay, using combinations of three inhibitors in accord with the invention.

FIG. 5 shows the effect of combinations of three inhibitors in accordance with the invention, using concentrations that do not produce an inhibiting effect per se on cell proliferation of a breast cancer cell line, measured by MTT assays and incorporation of Bromodeoxyuridine.

FIG. 6 shows the inhibiting effect on cell proliferation on another breast cancer cell line (Hs578T), measured by MTT assay, using combinations of three inhibitors in accordance with the invention.

FIG. 7 shows the inhibiting effect on cell proliferation using concentrations that do not produce an inhibiting effect per se on cell proliferation of a colon cancer cell line (SW620), measured by MTT assay, using combinations of two and three inhibitors in accordance with the invention.

FIG. 8 shows microphotographs of an experiment in closing of wounds.

### DETAILED DESCRIPTION OF THE INVENTION

The present invention foresees a pharmaceutical composition to inhibit cell proliferation that comprises a synergic combination of: i) an acyl-CoA-synthetase enzyme inhibitor (AcsI4), ii) a compound having an inhibiting effect on enzymes with cyclooxygenase activity (COX), and iii) a compound selected from a 5-lypoxygenase enzyme (LOX-5) inhibitor undo leukotriene receptor antagonist. The invention also foresees a method for treating and/or preventing cancer in a patient who has need of such treatment, that comprises administering to the patient a synergic combination of: i) an acyl-CoA-synthetase enzyme inhibitor (AcsI4), ii) a compound having an inhibiting effect on enzymes with cyclooxygenase activity (COX), and iii) a compound selected from a 5-lypoxygenase enzyme (LOX-5) inhibitor and a leukotriene receptor antagonist.

In accordance with a preferred embodiment, the compounds having an inhibiting effect on the acyl-CoA-synthetase enzyme (AcsI4) that can be used in the composition and in the methods of the present invention can be compounds derived from thiazolidinedione such as, but not limited to, Rosiglitazone (Rosi), Pioglitazone, Troglitazone (Trog), CS-011, AD-5075, BRL-49653, AY-31637, MCC-55, Darglitazone, Englitazone, Ciglitazone, and 5-(4-)(2-(methyl-2-pyridylamine)etoxi)-phenol) methyl)-2,4-thiazolidinediones.

In accordance with a preferred embodiment, the cyclooxygenase inhibitors that can be used in the composition and in the methods of the present invention are selected from the family of drugs called nonsteroid anti-inflammatories (NSAIDs) that comprise among others, but without being limited to these, aspirin acetylsalicylic acid), Diclofenac, Naproxen, Ibuprofen (Ibu), Indomethacin, Piroxicam, Ketoprofen, Sulindac and selective inhibitors of the cycloxygenase-2 (COX-2) enzyme—Nimesulide, Etodolac, Methoxycan, Celecoxib, Rofecoxib, Valdecoxib, Eteroxib (Eto) and structurally related compounds.

Also in accordance with a preferred embodiment, the compounds having an inhibiting effect on the 5-lypoxygenase (LOX-5) enzyme that can be used in the composition and in the methods of the present invention can be selected, without being limited to, Docebenone (AA-861), BW-7.55C, and Lonapalene (RS-43179), TMK-688 (Linazolast), DuP-654, R-68151 and E-6080, compounds that act by redox reaction, such as BW-755C, ICI-207968 and A-53612. Other LOX-5 inhibitors that can be used are compounds derived from Piperazinyl-R-8.5355 (2-(2-(4-chlorophenyl)-2-oxoethyl)-2,4-dihydro-4-(4-(4-hydroxyphenyl)-1-piperazinyl)-5-methyl-3H-1,2,4-triazol-3-one); and R-68151 ((1-ethyl-3 [4-(4-4-[4-hydroxyphenyl])-1-piperazinyl)phenyl]5-5-dimethyl)-2-thioxo-4-imidazolidinone)). In the family of iron chelating inhibitors such as derivates of N-hydroxyurea and hydroxamic acids, are Zileuton (Zil) (A-64077) and Atreleuton (ABT-761), derivatives of Zileuton like A78773, A79175 and A-76745 (Feuleuton).

Also in accordance with a preferred embodiment, the compounds having an inhibiting effect on the 5-lypoxygenase (LOX-5) enzyme that can be used in the composition and in the methods of the present invention can be selected from compounds derived from N-acetylhydroxylamine like BW-A4C and A63162 and other compounds that form enantiospecific interactions with the LOX-5 enzyme, that is, derivatives of methoxyalkylthiazols and methoxytetrahydropyrans, such as ZD-2138, ZM-230487 and hybrid molecules of natural products; derivatives of ZD-2138 such as L-697198, L-708780, L-739010 and L-746,530, derivatives of thiopyranilindols L-689065. L-691816 and L-699,333; compounds derived from dihydrobenzofuran-N-hydroxyurea like SB-210661 and SB-202235; heterocyclic compounds derived from N-hydroxyurea like CGS-26529 and CGS 23885, Also FLAP (5-Lypoxygenase-Activating Protein, 5-lypoxygenase activator protein) antagonistic compounds can be used such as MK-866, MK-0591 and Bay-X-1005. Other inhibiting compounds of both LOX-5 and LOX-12, such as E-3040 and CV-6505, can be used in the present invention.

Compounds that combine both the inhibition activities on LOX-5 and cyclooxygenases and also can be used in the compositions and the methods of the present invention, but not limited to these, are Darbufelone, S-2474 and Tebufelone, that are derived from a general chemical structure 2,6-di-ter-butyl-1-hydroxybenzene. And also Tepoxiline or RWJ-20485, RWJ-63556, Licofelone (ML-3000), Flobufen (VUFB-16066) and CMI-392; derivatives of Pyrazoline and modified derivatives of NSAIDs like DHDMBF and the PGV-20229.

In accordance with a preferred embodiment, the antagonistic compounds of leukotriene receptors that can be used in the compositions and the methods of the present invention include, but are not limited to, Zafirlukast (Zaf), Pranlukast, Montelukast and Pobilukast, ICI-198615, MK-571, SR-260, RG-12525, WY-48252, SKF-106203, L-649923, FPL-55712, the most preferred being Zafirlukast.

The compositions and the methods of the present invention can be used for treating or preventing proliferative diseases, such as cancer and psoriasis. In particular, they can be used for treating, and preventing breast cancer, colorectal cancer, esophagal cancer, stomach cancer, hepatocarcinoma, laryngeal cancer, ovarian cancer, epithelial ovarian cancer, pancreatic cancer, spleen cell carcinoma, squamous cell carcinoma, malignant melanoma, prostatic cancer, small cell (microcytic) and non-small cell lung cancer, renal cell carcinoma, testicular cancer, thyroid cancer, uterine sarcoma, endometrial sarcoma, cervical cancer, bladder cancer, osteosarcoma, chondrosarcoma, Ewing sarcoma, leukemia, lymphoma, multiple myeloma, gliomas, and other types of cancer.

Below are supplied some definitions intended to contribute to a better comprising of the description, although not meaning to limit in any way the scope of the present invention.

For a more concise description, some of the quantitative expressions given here are not qualified with the term “approximately.” it is understood that, whether the term “approximately” is explicitly used or not, each quantity presented here is intended to refer to the real value given and is also intended to refer to an approximation of such given value, which would reasonably be inferred based on common knowledge in the art, including approximations due experimental or measurement conditions for such value given.

In the description and the claims, the word “comprises” and variations of the word such as “comprising” and “they comprise” are not intended to exclude other additives, components, wholes or stages. Consequently, in the context of this description, the expressions “that comprise” or “comprising” mean “that mainly, but not necessarily only, includes,”

The term “composition” as it is used here comprises a product that comprises specified principles in predetermined quantities or proportions, as well as any product that results, directly or indirectly, from a combination of specified principles in specified quantities. The expression “therapeutically effective quantity” as used here refers to a quantity of a therapeutical agent to treat a treatable condition by administration of a composition of the invention. This quantity is the quantity sufficient to show a therapeutical or ameliorative response in a tissue, animal or human system. The effect can include, for example, treating the conditions listed here. The precise effective quantity for an individual will depend on the size and health of the individual, the nature and degree of the condition that is being treated, recommendations of the attending physician (investigator, veterinarian, doctor of medicine or other physician) and therapeutical agents or combination of therapeutical agents selected for administration.

As used here, a “pharmaceutical composition” refers to a product that comprises one or more act ve principles and an optional carrier that comprise inert ingredients, as well as any product that results, directly or indirectly, from the combination, complexing or aggregation of any two or more ingredients, or from the dissociation of one or more of the ingredients, or from other types of reactions or interactions of one or more of the ingredients. In general, the pharmaceutical compositions are prepared by uniformly and intimately associating the active principle(s) with a liquid carrier or a finely divided solid carrier or both, and later, if the case, conforming the product in the desired formulation. In particular, according to the present invention, each active principle could be formulated with a suitable carrier, and after that, if the case, to combine the formulations to form a single final preparation. Alternatively, each active principle can be formulated with a suitable carrier so as to form separate individual preparations, in order to administer them in a simultaneous or sequential way.

The pharmaceutical composition includes active compound sufficient to produce the effect desired on the progress or state of the disease. Therefore, the pharmaceutical compositions of the present invention comprise any composition prepared by mixing active compound(s) and at least one pharmaceutically acceptable carrier. By “pharmaceutically acceptable” is meant that the carrier, diluent or excipient must be compatible with the other ingredients of the formulation and must not be harmful for its recipient.

The term “treatment” as it is used here refers to any treatment of a condition or human disease and includes: (1) inhibiting the disease or condition, that is, deterring its development, (2) alleviating the disease or condition, that is, causing the regression of the condition, or (3) deterring the symptoms of the disease.

The term “to inhibit” includes its generally accepted meaning that includes “to restrict,” “to alleviate.” “to improve,” and “to slow,” “to deter or to invert the progression, severity or a resulting symptom.” As used here, the expression “medical therapy” includes in vivo or ex vivo diagnostic and therapeutic systems carried out in humans.

In general, the pharmaceutical compositions of the present invention can be administered by standard routes, such as by parenteral route (for example, intravenous, intravertebral, subcutaneous or intramuscular), oral, tracheal, bronchial, intranasal, pulmonary, buccal, rectal, transdermal or topical. The administration can be systemic, regional or local.

The types of pharmaceutical compositions that can be used include: tablets or pills, chewable tablets, capsules (including microcapsules), powders, powders for reconstitution, solutions, parenteral solutions, aerosol solutions, ointments (creams and gels), suppositories, suspensions, and other types described here or that are evident for an expert in the field, from general knowledge of the art. The active principle(s), for example, can also be in the form of a complex including cyclodextrins, their ethers or their esters.

The inhibiting compounds used in the present invention can be taken in suitable forms for administration by common processes, using auxiliary substances such as liquid or solid ingredients, in powder, such as pharmaceutically usual liquids or solids and expanders, solvents, emulsifiers, lubricants, flavoring agents, pigments and/or buffering substances (buffers). The auxiliary substances frequently used include magnesium carbonate, titanium dioxide, lactose, saccharose, sorbitol, mannitol and other sugars or sugar alcohols, talcum, lactoprotein, gelatin, starch, amylopectin, cellulose and its derivatives, animal and vegetable oils such as fish liver oil, sunflower, peanut or sesame, polyethylene glycol; and solvents such as sterile water and mono- or polyhydric alcohols such as glycerol; as well as disinfecting agents and lubricating agent such as magnesium stearate, calcium stearate, sodium stearyl fumarate and polyethyleneglycol waxes. The mixture can then be processed into granules or compressed into tablets.

The active principles can be separately premixed the other nonactive principles, before being mixed to form a formulation or, alternatively, the active principles can be mixed together, before being mixed with the nonactive principles to form a formulation.

Soft gelatin capsules can be prepared with capsules that contain a mixture of the active principles of the invention, vegetable oil, fat, or other vehicles suitable for soft gelatin capsules. Hard gelatin capsules can contain granules of the active principles. Hard gelatin capsules can also contain the active principles with solid ingredients in powder, such as lactose, saccharose, sorbitol, mannitol, potato starch, corn starch, amylopectin, cellulose derivatives or gelatin.

Units for rectal administration can be prepared (i) in the form of suppositories that contain the active substances mixed with a base of neutral fat; (ii) in the form of a rectal gelatin capsule that contains the active substance in mixture with a vegetable oil, paraffin oil or another vehicle suitable for rectal gelatin capsules; (iii) in the form of a ready-to-use microenema; or (iv) in the form of a dry microenema formulation to be reconstituted in a suitable solvent before its administration.

Liquid preparations can be prepared in the form of boiled syrups, elixirs, drops or concentrated suspensions, for example, solutions or suspensions that contain the active principles and the remainder consists of for example, sugar or sugar alcohols, and a mixture of ethanol, water, glycerol, propylene glycol and polyethylene glycol, if desired, such liquid preparations can contain pigment agents, flavoring agent, preservatives, saccharin and carboxymethylcellulose and other thickening agents. Liquid preparations can also be prepared in dry powder form, reconstituted with suitable solvent before their use. Solutions for parenteral administration can be prepared as the solution of a formulation of the invention in a pharmaceutically acceptable solvent, such as a sterile water solution or nonwater solvent, as vegetable oil, esters of long-chain aliphatic acids or propylene glycol. These solutions can also contain stabilizers, preservatives and/or buffers. Solutions for parenteral administration can also be prepared as a dry preparation, reconstituted with a suitable solvent before their use.

The compositions of the invention can be prepared in the form of ointments (creams or gels) to be applied topically on the skin or the scalp. In an embodiment of the invention, an oil emulsion is prepared in water to form a cream. The active compounds in powder form are dissolved in a suitable solvent, such as, for example, propylene glycol. The aqueous phase can alternatively include an alcohol or isopropanol, adding a thickener, for example, Carbomer 934 or 940. The oily phase preferably includes mineral oil, petroleum jelly, cetyl alcohol and/or stearyl alcohol. Emulsifiers can be used such as polysorbate 80, sorbitan monostearate or others known in the art. There can be added buffering agents, antioxidants and chelating agents to improve the characteristics of the formulation.

Preparations for topical administration can be prepared for delivery in an aerosol. In these cases, the inhibiting compounds can be mixed with known excipients for aerosol, such as saline solution, alcohol, or fatty acid derivatives, to enhance bioavailability if necessary.

Formulations are also supplied in accordance with the present invention and “part kits” that comprise one or receptacles that contain separately one or more of the ingredients of the pharmaceutical composition of the invention in a suitable carrier, for its coadministration in a combination therapy. Various written materials can be associated with these receptacles, such as instructions for use, or a notification in the form prescribed by a governmental agency that governs the manufacture, use or sale of pharmaceutical products, whose notification reflects approval by the agency of the manufacture, use or sale for human administration.

The phrases “combination therapy” or “coadministration” are intended to embrace the administration of each active agent in a sequential way in a system that will provide the beneficial effects resulting from the combination of drugs, and it is intended to embrace the coadministration of these agents in a substantially simultaneous way, such as in a single dose unit that has a fixed ratio of these ingredients, or in multiple dose units, separate for each active agent.

The quantity of each active principle to be administered and the dosage system to treat a disease condition with the compounds and compositions of the invention depend on a variety on factors, including age, weight, sex and medical condition of the patient, the severity of the disease and the route and frequency of administration, as well as the particular compound employed, so that it can vary widely.

Depending on the compound employed, the compositions of the invention can contain as an acyl-CoA-synthetase enzyme inhibitor (AcsI4), for example, Troglitazone in the range of 1-600 mg, more precisely, in the range of 5-400 mg and more specifically 50-200 mg, per dose unit; or, for example, Rosiglitazone in the range of 0.01-20 mg, more precisely 0.1-5 mg and more specifically 0.5-2 mg per dose unit.

Additionally, the compositions of the invention can contain as enzyme inhibitor compound with cyclooxygenase (COX) activity, for example, Ibuprofen in the range of 1-1000 mg, more precisely 10-800 mg and more specifically 100-400 mg, per dose unit; or, for example, Etoricoxib in a range of 1-200 mg, more precisely 5-120 mg, and more specifically 10-60 mg per dose unit.

The compositions of the invention additionally contain a compound selected from a 5-lipoxygenase enzyme (LOX-5) inhibitor and a leukotriene receptor antagonist. As an example of an LOX-5 enzyme inhibitor, the composition can contain Zileuton, in a range of 1-2000 mg, more precisely 50-1200 mg, and more specifically 100-600 mg, per dose unit. As an example of a leukotriene receptor antagonist compound the composition can contain Zafirlukast in a range of 0.1-100 mg, more precisely 1-20 mg and more specifically 5-10 mg, per dose unit.

In accordance with preferred embodiments of the composition of the invention, the following active compound combinations and their quantity (in mg) are provided below, present per dose unit, for different dose levels, low, average and high.

| | Dose (quantities in mg) | | |
|---|---|---|---|
| Combinations | Low | Average | High |
| Troglitazone | 150.0 | 170.0 | 200.0 |
| Zafirlukast | 1.0 | 5.0 | 10.0 |
| Ibuprofen | 100.0 | 200.0 | 400.0 |
| Troglitazone | 150.0 | 170.0 | 200.0 |
| Zileuton | 200.0 | 300.0 | 600.0 |
| Ibuprofen | 100.0 | 200.0 | 400.0 |
| Troglitazone | 150.0 | 170.0 | 200.0 |
| Zileuton | 200.0 | 300.0 | 600.0 |
| Etoricoxib | 15.0 | 30.0 | 60.0 |
| Troglitazone | 150.0 | 170.0 | 200.0 |
| Zafirlukast | 1.0 | 5.0 | 10.0 |
| Etoricoxib | 15.0 | 30.0 | 60.0 |
| Rosiglitazone | 1.5 | 1.7 | 2.0 |
| Zafirlukast | 1.0 | 5.0 | 10.0 |
| Ibuprofen | 100.0 | 200.0 | 400.0 |
| Rosiglitazone | 1.5 | 1.7 | 2.0 |
| Zileuton | 200.0 | 300.0 | 600.0 |
| Ibuprofen | 100.0 | 200.0 | 400.0 |
| Rosiglitazone | 1.5 | 1.7 | 2.0 |
| Zileuton | 200.0 | 300.0 | 600.0 |
| Etoricoxib | 15.0 | 30.0 | 60.0 |

By way of example and not limitative, below are given various pharmaceutical formulations that comprise the composition of the invention.

### Formulation 1

### Single Daily Dose Formulation

| Component | Quantity (mg) |
|---|---|
| Troglitazone | 150.0 |
| Zafirlukast | 1.0 |
| Etoricoxib | 15.0 |
| Lactose | 146.0 |
| Microcrystalline cellulose | 64.0 |
| Croscarmellose sodium | 8.0 |
| PVP K30 | 10.0 |
| Talcum | 12.0 |
| Magnesium stearate | 4.0 |
| Total | 410.0 |

Volatile components:
Ethyl alcohol: 50 mg

### Water: 50 mg

### Method of Preparation

PVP K30 is dissolved in ethyl alcohol and water. Separately, Zafirlukast is mixed with Etoricoxib and microcrystalline cellulose for 15 minutes. Then lactose is added, half of the indicated quantity of croscarmellose sodium and Troglitazone. It is mixed for 15 minutes more and is placed in a roto granulator. The solution of PVP K30 in water and alcohol is slowly added. It is dried at 45° C. to a residual humidity of less than 2%. It is granulated in a rocking mill through 16 mesh. The remaining quantity of croscarmellose sodium, or talcum and/or magnesium stearate is mixed for 5 minutes and compressed to theoretical weight of 410 mg and hardness of 10±2 KP.

### Formulation 2

### 3 Daily Dose Formulation

| Component | Quantity (mg) |
|---|---|
| Rosiglitazone | 1.5 |
| Zileuton | 200.0 |
| Ibuprofen | 100.0 |
| Lactose | 148.5 |
| Microcrystalline cellulose | 75.0 |
| Croscarmellose sodium | 10.0 |
| PVP K30 | 10.0 |
| Talcum | 15.0 |
| Magnesium stearate | 5.0 |
| Total | 565.0 |

### Volatile Components:

Ethyl alcohol: 70 mg

### Water: 70 mg

### Method of Preparation

PVP K30 is dissolved in ethyl alcohol and water. Rosiglitazone is mixed separately with microcrystalline cellulose for 15 minutes. Then lactose is added, half of the indicated quantity of croscarmellose sodium, Zileuton and Ibuprofen. It is mixed for 15 minutes more and is placed in a roto granulator. The solution of PVP K30 in water and alcohol is slowly added. It is dried at 45° C. to a residual humidity of less than 2%. It is granulated in a rocking mill through 16 mesh. The remaining quantity of croscarmellose sodium, talcum and magnesium stearate is mixed for 5 minutes and compressed to theoretical weight of 565 mg and hardness of 14±2 KP.

### Formulation 3:

### Single Daily Dose “PA” Formulation (*)

| Component | Quantity (mg) |
|---|---|
| Rosiglitazone | 2.0 |
| Zileuton | 600.0 |
| Ibuprofen | 400.0 |
| HPMCK100M | 48.0 |
| HPMC K4M | 60.0 |
| Microcrystalline cellulose | 75.0 |
| Talcum | 30.0 |
| Magnesium stearate | 5.0 |
| Total | 1220.0 |

### (*) PA: Prolonged Action

### Volatile Components:

### Water: 300 mg

### Method of Preparation

Rosiglitazone, Zileuton, ibuprofen, microcrystalline cellulose, HPMC K100 M and HPMC K4 M are mixed for 20 minutes in a roto granulator. Water is slowly added and it is mixed for 20 minutes more. The moist mixture is dried at 45° C. to a residual humidity of less than 1.5%. It is granulated in a rocking mill through 14 mesh. It is mixed with talcum and magnesium stearate for 5 minutes and is compressed to theoretical weight of 1220 mg and hardness of 20±3 KP.

The following examples are given for the purpose of demonstrating and illustrating certain embodiments and preferred aspects of the present invention, and they should not be considered as limiting of its scope.

### EXAMPLES

In the experiments described below, the cells used are human breast and colon cancer cell lines. The breast cancer cell lines were obtained from Lombarda Cancer Center, Georgetown University Medical Center. The breast cancer lines are cell lines called MDA-MB-23 and Hs578T. These cells show a high rate of proliferation, absence of estrogen receptors, are capable of forming tumors in naked mice and show a highly aggressive phenotype. Both lines are used very frequently as breast cancer models. The colon cancer cell line is the cell line called SW620 that was originally isolated from the lymphatic nodules of a patient with colon adenocarcinoma.

The cells are cultivated in polystyrene culture bottles (Corning, Corning, N.Y.). Both cell lines are kept in culture in DMEM medium (Dulbeco's Modified Tagle Medium, obtained from Gibco, Invitrogen Corporation, NY. USA) and supplemented with 10% fetal bovine serum (FBS) (obtained from PAA, GmbH Laboratories, Pasching, Austria), and supplemented with 100 U/ml of penicillin and 10 μg/ml of streptomycin (Gibco, Invitrogen Corporation, NY, USA) in incubator 37° C. In humidified atmosphere of 95% O₂/5% CO₂.

There were carried out curves of dose-cell proliferation inhibition response of the human breast cancer cell line MDA-MB-231, with the intent of objectively knowing the minimum concentrations that produce a proliferation inhibiting effect.

In all the cases, the cell proliferation measurement was carried out using the MTT techniques of incorporation of Bromodeoxyuridine (BrdUr) (5-bromine-2′-deoxyuridine of Elisa Roche Applied Science, Mannheim, Germany) and the violet crystal coloration technique. The results were comparable with the use of the three methodologies, the MTT and BrdUr assays being the most sensitive.

### Example 1

### Determination of the Cell Proliferation Minimum Inhibitory Dose, of Inhibitors Supplied in Isolated Form, by Means of the MTT Assay

The cells were cultivated in microplates (Orange Scientific) of 96 cavities in the amount of 4,000 cells per cavity in 200 ul of DMEM supplemented with FCS supplemented with 100 U/ml of penicillin and 10 ug/ml of streptomycin in incubator at 37° C. with atmosphere of 95% O₂/5% CO₂. A the end of this period, the cells were cultivated for another 24 hours in culture medium in the absence of serum. At the end of this period, culture medium with scram in the absence (control) or presence of different inhibitors was added, in accordance with the following:

### Troglitazone: 5 μM, 25 μM, 50 μM, 75 μM, 100 μM

### Rosiglitazone: 5 μM, 25 μM, 50 μM, 75 μM, 100 μM

### Ibuprofen: 10 μM, 100 μM, 250 μM, 500 μM, 1000 μM

Etoricoxib: in M, 10 nM, 100 nM, 1000 nM, 10000 n′A4

### AA861 (Doceberione): 1 μM, 5 μM, 10 μM, 20 μM

### Zileuton: 10 μM, 100 μM, 250 μm, 500 μM

### Zafirlukast: 0.1 nM, 1 mM, 10 nM, 100 nM.

The cells were kept in culture for 72 hours. Then, thiazol blue bromide reagent was added (3-(4,5-dimethylthiazol-241)-2,5-diphenyltetrazolium bromide). It was incubated for 180 minutes at 37° C. After this time, the culture medium was discarded and the formazan crystals formed were dissolved with 200 ul of dimethylsulfoxide. The microplates were agitated for 15 minutes to dilute the crystals of homogeneous form, and absorbance was determined at 620 nm in a plate reader, “Biotra II, plate reader,” of Amersham Biosciences Inc., Piscataway, N.J., USA.

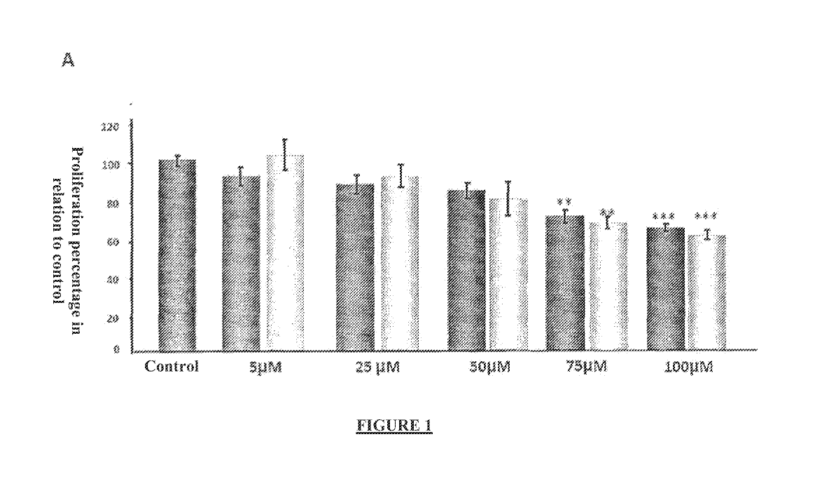

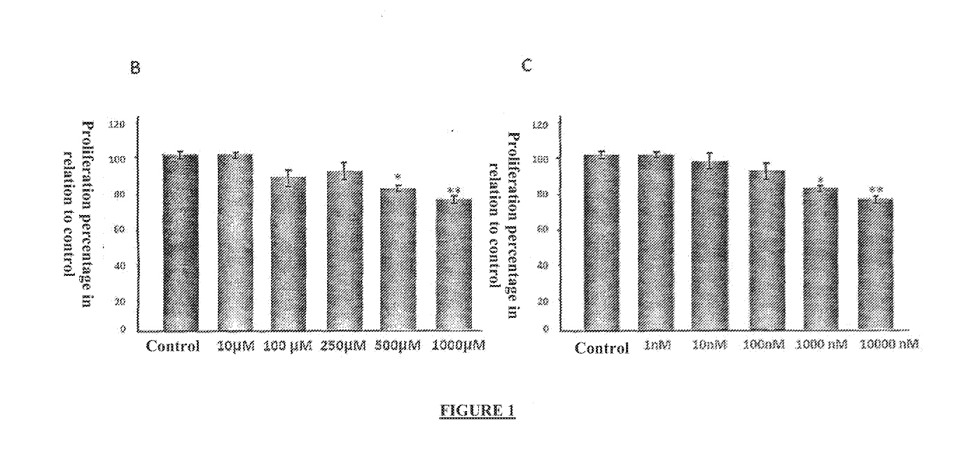

In FIG. 1 are also shown the results expressed as growth percentage in relation to the control, carrying out a minimum of 5 times by treatment in three different experiments. The values are the average value±the standard deviation of three individual experiments. The values are significantly different from the control ** (p#0.05), *** (p#0.01).

It is observed in FIG. 1A that the minimum inhibitory concentration is 75 μM for two compounds having an inhibiting effect on the acyl-CoA-synthetase enzyme (AcsI4), the thiazolidinediones Troglitazone (dark bars) and Rosiglitazone (light bars).

NSAIDs compounds significantly inhibited proliferation in concentrations of 500 μM for the Ibuprofen and 1000 nM for Etoricoxib. (FIG. 1B and FIG. 1C, respectively).

In FIG. 1D and FIG. 1E, it is observed that the minimum proliferation inhibitory concentration for the compounds AA861 and Zileuton, pertaining to the LOX family of inhibitors, was 10 μM and 500 μM, respectively. Concentrations of up to 100 nM were not sufficient to inhibit cell proliferation when the action inhibitor of lipoxygenate products, Zafirlukast, was used (FIG. 1F).

### Example 2

### Effect on Cell Proliferation of Cell Line MDA-MB-231 Measured by MTT Assay, by Combining Pairs of Inhibitors in Concentrations that do not have an Effect Per Se

The cells were cultivated as described in Example 1, After that, the culture medium with serum was added in the absence (control) or presence of combinations of pairs of different inhibitors in per se noninhibiting concentrations. The data of the per se noninhibiting concentrations are those obtained from the experiment of Example 1, namely:

Experiment A: Combination of LOX inhibitors/COX inhibitors

### AA861 (5 μM)/Ibuprofen (10 μM)

### Zileuton (10 μM)/Ibuprofen (10 μM)

### AA0.861 (5 μM)/Etoricoxib (10 nM)

### Zileuton (10 μM)/Etoricoxib (10 nM)

Experiment B: Combination of LOX inhibitors/acyl-CoA-synthetase inhibitors

### AA861 (5 μM)/Troglitazone (5 μM)

### Zileuton (10 μM)/Troglitazone (5 μM)

### AA861 (5 μM)/Rosiglitazone (5 μM)

### Zileuton (10 μM)/Rosiglitazone (5 μM)

Experiment C: Combination of acyl-CoA-synthetase inhibitors/COX inhibitors

### Troglitazone (5 μM)/Ibuprofen (10 μM)

### Troglitazone (5 μM)/Etoricoxib (10 nM)

### Rosiglitazone (5 μM)/Ibuprofen (10 μM)

### Rosiglitazone (5 μM)/Etoricoxib (10 nM)

Experiment D: Combination of leukotriene receptor antagonists with acyl-CoA-synthetase inhibitors or with COX inhibitors or LOX inhibitors
Zafirlukast (100 nM)/Troglitazone (5 μM)
Zafirlukast (100 nM)/Rosiglitazone (5 μM)
Zafirlukast (100 nM)/AA861 (5 μM)
Zafirlukast (100 nM)/Zileuton (10 μM)
Zafirlukast (100 nM)/ibuprofen (10 mM)
Zafirlukast (100 nM)/Etoricoxib (10 nM)

The cells were kept in culture for 72 hours, and the determination of the percentage of cell proliferation inhibition was done in accordance with the method described in Example 1.

The results are expressed as growth percentage in relation to the control, carried out a minimum of 5 times by treatment in three different experiments. The values are the averages±the standard deviation of three individual experiments. The values are significantly different from the control ** (p#0.05), *** (p#0.001).

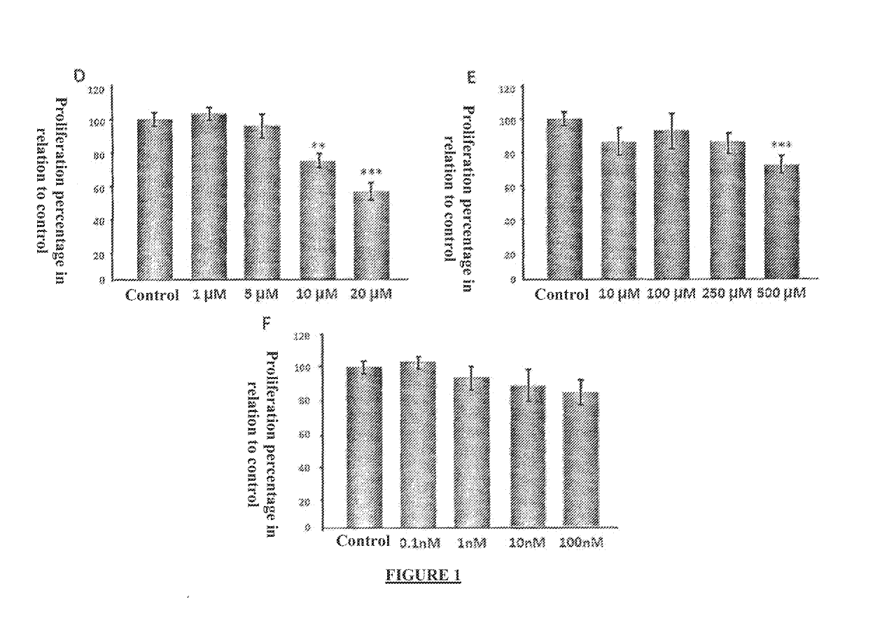

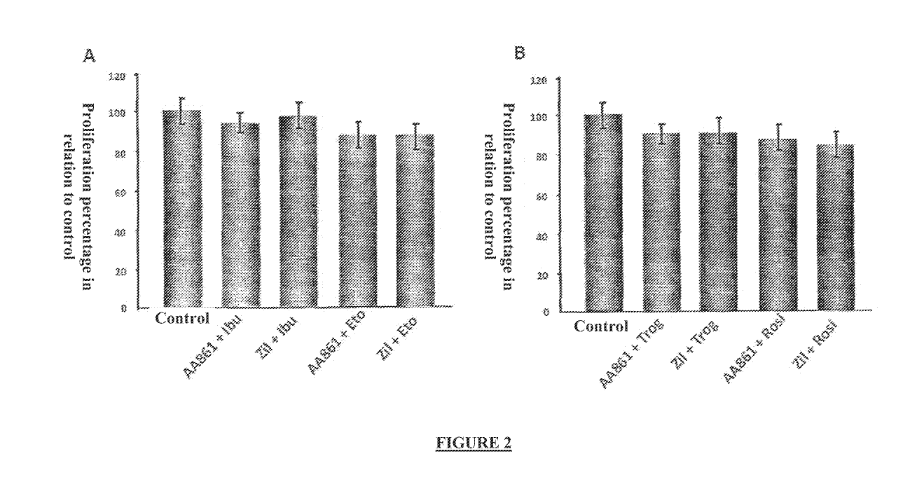

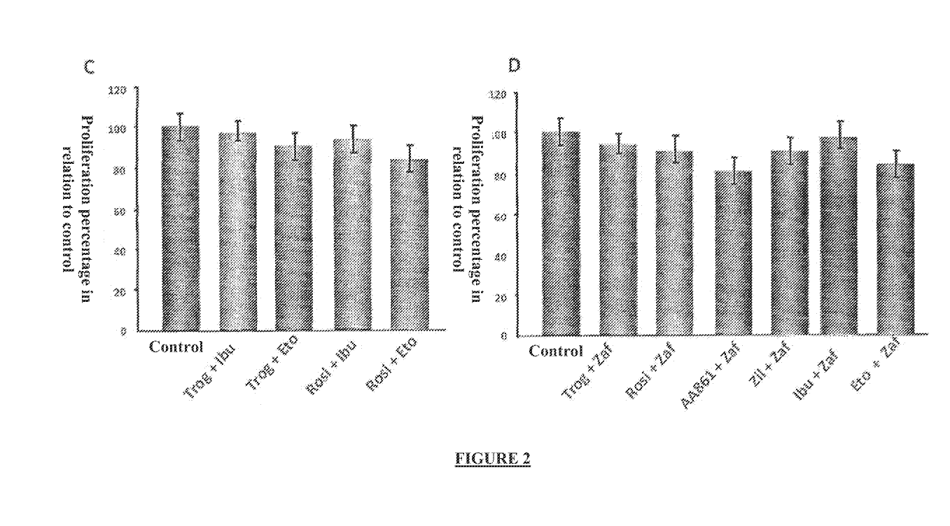

FIG. 2A shows the combination of COX enzyme inhibitors with LOX inhibitors. FIG. 2B shows the combination of LOX inhibitors with acyl-CoA-synthetase inhibitors. FIG. 2C shows the combination of acyl-CoA-synthetase inhibitors with COX inhibitors. FIG. 2D shows the combination of pairs of: inhibitors of the action of lipoxygenated products (leukotriene receptor antagonist) with acyl-CoA-synthetase inhibitors; with COX inhibitors and LOX inhibitors.

In none of the combined treatments of pairs of inhibiting compounds in accordance with what was previously detailed and using concentrations that do not have inhibiting effect per se, significant inhibition of cell proliferation can be observed.

### Example 3

### Effect on Cell Proliferation of Cell Line MDA-MB-231 Measured by MTT Assay, by Combining Pairs of Inhibitors Used in Minimum Concentrations that Produce an Inhibiting Effect

The cells were cultivated as was described in Example 1. After that, the culture medium with serum was added in the absence (control) or presence of combinations of pairs of different inhibitors used in the experiment of Example 1, using the minimum concentrations that resulted in the inhibition of proliferation in that example, namely:

### Troglitazone (75 μM)/Ibuprofen (500 μM)

### Troglitazone (75 μM)/Zileuton (500 μM)

### Troglitazone (75 μM)/Etoricoxib (1000 nM)

### Troglitazone (75 μM)/Zafirlukast (100 nM)

### Zileuton (500 μM)/Zafirlukast (100 nM)

### Ibuprofen (500 μM)/Zafirlukast (100 nM)

### Zileuton (500 μM)/Etoricoxib (1000 nM)

The cells were kept in culture for 72 hours, and the determination of the percentage of cell proliferation inhibition was done in accordance with the method described in Example 1.

The results are expressed as growth percentage in relation to the control, carried out a minimum of 5 times by treatment in three different experiments. The values are the averages the standard deviation of three individual experiments. The values are significantly different from the control ** (#0.05), *** (p#0.001).

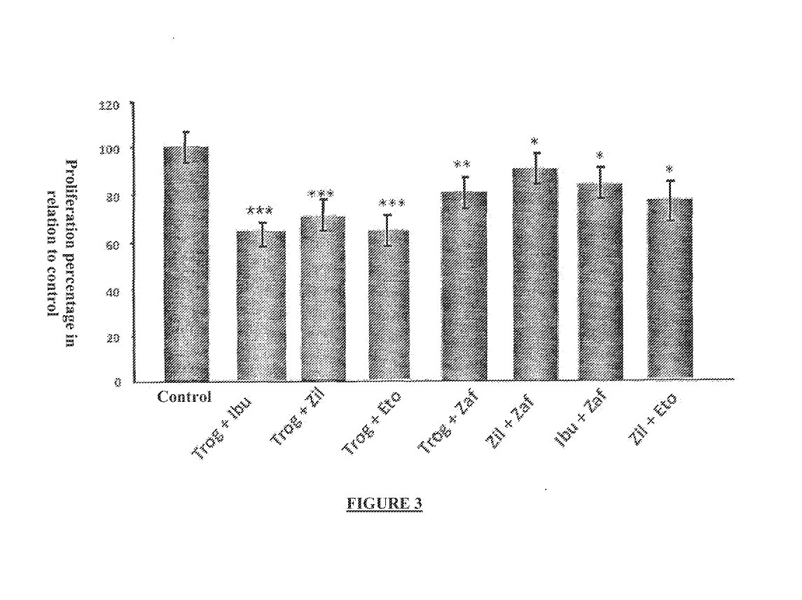

As observed in FIG. 3, when distinct combinations of pairs of inhibitors were used, using the minimum concentrations that produce an effect on cell proliferation, there was observed a significant inhibition. In particular, the percentage of inhibition of proliferation with the use of all combinations of pairs of inhibitors experienced an increment as to the inhibition observed when the inhibitors were used individually. This increment did not reach the cumulative of the individual inhibitions in any of the combinations used. The combination of the drugs Troglitazone (acyl-CoA-synthetase inhibitor) and Zileuton (LOX inhibitor) was the one that reached an inhibition close to the cumulative values. The combination of Zileuton (a LOX inhibitor) with Zafirlukast (inhibitor of the action of lipoxygenated products) was the least effective combination.

### Example 4

### Effect on cell Proliferation of Cell Line MDA-MB-231 Measured by MTT Assay, by Combining Three Inhibitors in Concentrations that Per Se do not have an Inhibiting Effect

The cells were cultivated as described in Example 1. After that, the culture medium with serum was added in the absence (control) or presence of combinations of three different inhibitors in per se noninhibiting concentrations. The data of the per se noninhibiting concentrations are those obtained from the experiment of Example 1, namely:

### Troglitazone (5 μM)/AA681 (5 μM)/Ibuprofen (10 μM)

Troglitazone (5 μM)/Zafirlukast (100 nM)/Ibuprofen (10 μM)

### Troglitazone (5 μM)/Zileuton (10 μM)/Ibuprofen (10 μM)

### Troglitazone (5 μM)/Zileuton (10 μM)/Etoricoxib (10 nM)

Troglitazone (5 μM)/Zafirlukast (100 nM)/Etoricoxib (10 nM)

### Rosiglitazone (5 μM)/AA681 (5 μM)/Ibuprofen (10 μM)

Rosiglitazone (5 μM)/Zafirlukast (100 nM)/Ibuprofen (10 μM)

### Rosiglitazone (5 μM)/Zileuton (10 μM)/Ibuprofen (10 μM)

### Rosiglitazone (5 μM)/Zileuton (10 μM)/Etoricoxib (10 nM)

Rosiglitazone (5 μM)/Zafirlukast (100 nM)/Etoricoxib (10 nM)
Zileuton (10 nM)/ibuprofen (10 μM)/Zafirlukast (100 nM)
Zileuton (10 μM)/Etoricoxib (10 nM)/Zafirlukast (100 nM)

The cells were kept in culture for 72 hours, and the determination of the percentage of cell proliferation inhibition was done in accordance with the method described in Example 1.

The results are expressed as growth percentage in relation to the control, carried out a minimum of 5 times by treatment in three different experiments. The values are the averages±the standard deviation of three individual experiments. The values are significantly different from the control **(p#0.05), ***(p#0.001).

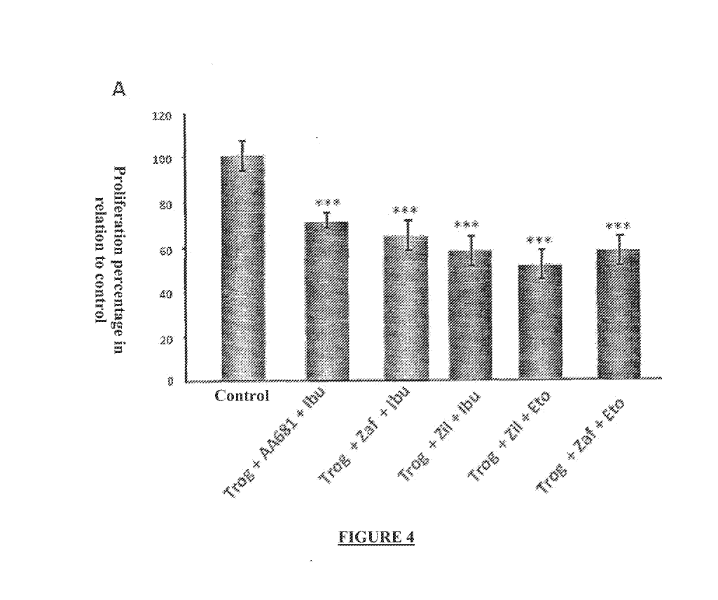

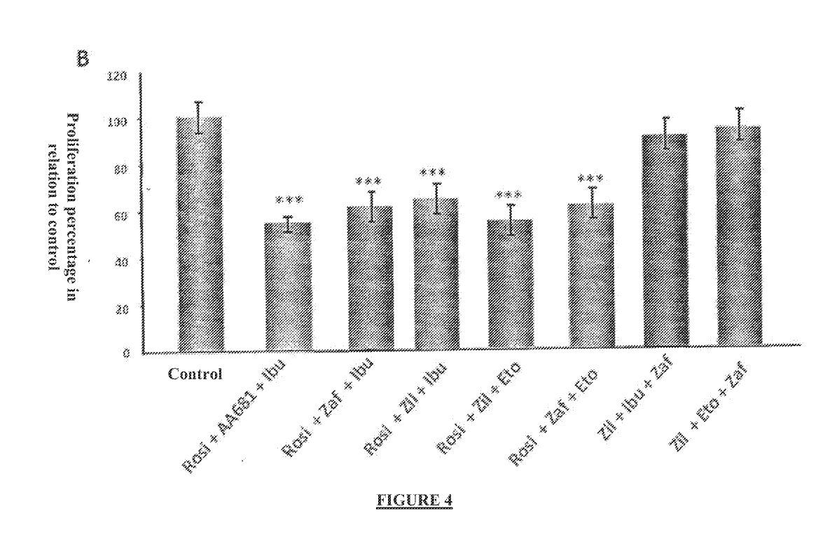

Surprising, when the combination of three inhibitors of different families in concentrations that per se do not have any effect was used, a significant inhibition of cell proliferation was observed. This effect is shown in FIG. 4A and FIG. 4B. The greatest inhibition was observed when the combination of Troglitazone/Zileuton/Etoricoxib was used (FIG. 4A), reaching an inhibition of 48.8%. This value also is higher than that obtained with combinations of two inhibitors in effective concentrations. It is noted that an inhibiting effect was not observed combining three inhibitors when an acyl-CoA-synthetase inhibitor was absent from the combination (see FIG. 4B, last two combinations: Zil+Ibu+Zaf or Zil+Eto+Zaf).

These results show clearly that only when the combination of three inhibitors if used, using an acyl-CoA-synthetase inhibitor in conjunction with a COX inhibitor and a compound selected from a LOX inhibitor and an inhibitor of the action of lipoxygenated products (leukotriene receptor antagonist), can a clear synergic effect be observed. This inhibiting effect in the combination of three inhibitors is observed in concentrations that do not have any effect when used in the isolated form or in a combination of two inhibitors. Moreover, the concentrations used in the ternary combination are some times less than the minimum effective doses, being in the case of members of the family of thiazolidinediones (acyl-CoA-synthetase inhibitors) more than 10 times less than the minimum effective concentrations, and being for members of the family of lipoxygenases inhibitors 50 times less than the minimum effective concentrations.

It must be emphasized that the presence of acyl-CoA-synthetase inhibitors is essential for observing the synergic effect when concentrations of the inhibitors that do not per se show any effect are used.

### Example 5

### Effect on Cell Proliferation of Cell Line MDA-MB-231, Measured by MTT Assay and BrdUr, Through the Combination of Three Inhibitors

The cells of cell line MDA-MB-231 were cultivated in microplates (Orange Scientific) of 96 cavities in the amount of 4,000 cells per cavity in 100 μl of DMEM supplemented with FCS supplemented with 100 U/ml of penicillin and 10 μg/ml of streptomycin in incubator at 37° C. with atmosphere of 95% O₂/5% CO₂. A the end of this period, the cells were cultivated for another 24 hours in culture medium in the absence of serum. After that, culture medium with serum was added in the absence (control) or presence of different inhibitors. The cells were kept in culture for 72 hours. After this period, a pulse of Bromodeoxyuridine BrdUr was added (10 μl to get a final concentration of 100 μM) of 2 hr duration. At the end of this period, the culture medium was removed, the cells were fixed with a solution called FixDenat (Roche) for 30 minutes. After that, 100 μl of anti-BrdU-POD was added through the cavity (1/10000 final dilution), incubating the cells for 90 minutes. After that, the cavities were washed with PBS 1× three times. A tetramethyl-benzidine solution was added as substratum and at the end of 10 minutes the reaction was interrupted by adding 25 μl of a 1 M solution of sulfuric acid for one minute with stirring, and then a colorimetric analysis was performed. The colorimetric analysis was done using the ELISA Plate Reader (Biotra II Plate Reader, Ammersham, Bioscience).

The combinations and the concentrations used were the same used in the experiments described in Example 4. In FIG. 5 are shown the results obtained (light bars) by means of the BrdUr method, and the combinations of three inhibitors used are described in the axis of these abscissas. Also in FIG. 5 are shown the results obtained (dark bars) by the MTT method (see Example 4) for the same combinations.

In particular, the combinations and concentrations used were the following:

### Troglitazone (5 μM)/Zileuton (10 μM)/Ibuprofen (10 μM)

Troglitazone (5 μM)/Zafirlukast (100 nM)/Ibuprofen (10 μM)

### Troglitazone (5 μM)/Zileuton (10 μM)/Etoricoxib (10 nM)

### Troglitazone (5 μM)/Zafirlukast (100 μM)/Etoricoxib (10 μM)

The results are expressed as growth percentage in relation to the control, carrying out a minimum of 5 times by treatment in three different experiments. The values are the averages±the standard deviation of three individual experiments. The values are significantly different from the control ** (p#0.05), *** (p#0.001).

As shown in FIG. 5, the results obtained by means of both MTT technique and incorporation of BrdUr were comparable and confirm the general conclusions obtained using the MTT technique (Example 4). These results show clearly that only when the combination of three inhibitors is used, using an acyl-CoA-synthetase inhibitor in conjunction with a COX inhibitor, and a compound selected from a LOX inhibitor and an inhibitor of the action of lipoxygenated products (leukotriene receptor antagonist), can a clear synergic effect be observed.

### Example 6

### Effect on Cell Proliferation of the Human Breast Cancer Cell Line Hs578T and Colon Cancer Cell Line SW620 Measured by MTT Assay, Through the Combinations of Three Inhibitors in Concentrations that Per Se do not have an Inhibiting Effect

The results of the preceding examples were corroborated using a second human breast cancer cell line Hs578T, with features similar to MDA-MB-231, and a colon cancer cell line SW620.

The sowing of the cells and the treatment with different inhibitors and the MTT assay were carried out as described in Example 1. The combinations of different inhibitors and the concentrations used were the same used in the described experiments in Example 4.

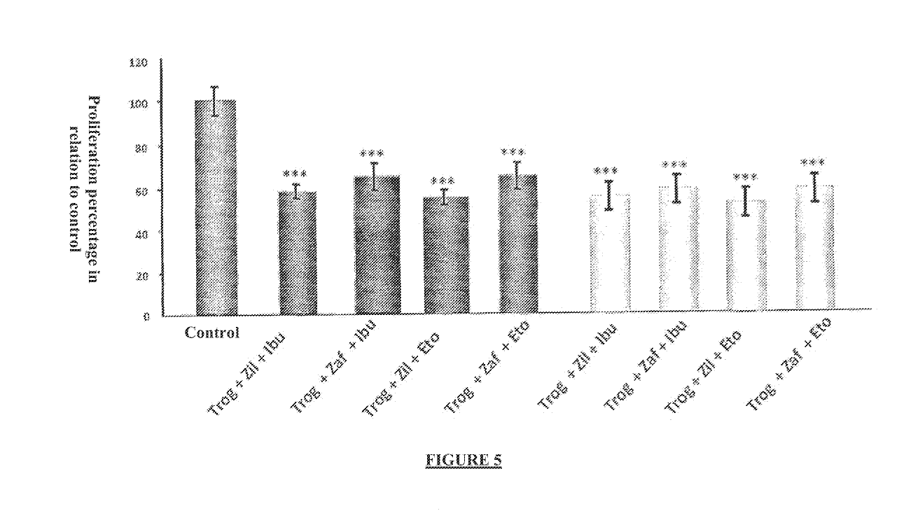

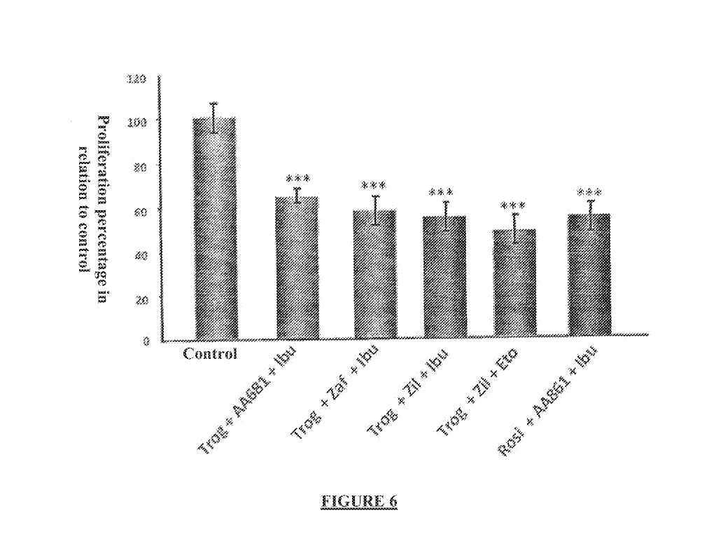

The results shown in FIG. 6 are expressed as growth percentage in relation to the control, carrying out a minimum of 5 times by treatment in three different experiments. The values are the averages±the standard deviation of three individual experiments. The values are significantly different from the control ** (p#0.05), *** (p#0.001).

In FIG. 6 are shown the results of the combination of three inhibitors in accordance with the invention in per se noneffective combinations. Significant inhibition of cell proliferation was observed (of Hs578T cells), that reached inhibition values of 45.5%, Again, an inhibiting effect was not observed when the combination of three inhibitors was carried out in the absence of an acyl-CoA-synthetase inhibitor (data not shown).

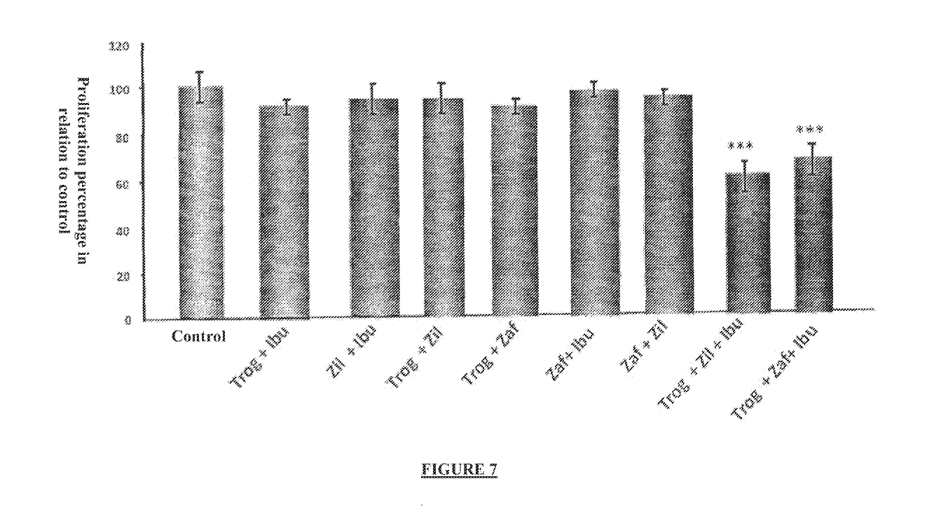

The results shown in FIG. 7 are expressed as growth ercentage in relation to the control, carrying out a minimum of 5 times by treatment in three different experiments. The values are the averages±the standard deviation of three individual experiments. The values are significantly different from the control ** (p#0.05), *** (p#0.001).

In FIG. 7 are shown the results of the combination of two and three inhibitors in accordance with the invention in per se noneffective combinations. In none of the combined treatments of pairs of inhibiting compounds using concentrations that do not have an inhibiting effect per se, significant inhibition of cell proliferation can be observed (of SW620 cells). However, significant inhibition of cell proliferation was observed when the combination of three inhibitors was used, that reached inhibition values of 39%. Again, inhibiting effect was not observed when the combination of three inhibitors was carried out in the absence of an acyl-CoA-synthetase inhibitor (data not shown).

### Example 7

### Experiment on Wounds for Measuring Cell Migration

Considering that the cell line MDA-MB-231 shows an aggressive phenotype, experiments were performed to see whether the effect of the different inhibitor combinations that was observed for cell proliferation was also observed on an aggressive phenotype of this line. Therefore, the migration capacity was used as a measure of the aggressiveness of the cell line, The migration was studied by means of the wound recovery experiment. In accordance with this experiment, breast cancer cells of line MDA-MB-231 were sown in a pane of 6 cavities, without arriving at confluence, at a density of 600,000 cells per cavity and kept in culture in DMEM/10% FBS medium plus the addition of 100 U/ml of penicillin and 10 μg/ml of streptomycin for 24 hours. After that, the cells were incubated with DMEM medium with antibiotic without serum for another 24 hours. After this incubation, culture medium with scram was added in the absence or presence of the different combinations of inhibiting compounds and was incubated for 48 hours, The cells were washed with PBS 1× and, with a sterile plastic tip for automatic pipette of 10 ul, a wound was made in cross form. Incubation was continued with the taking of photographs at different times. The wounds were photographed with a digital camera connected to an inverted Nikon Diafot 300 microscope with Normanski optics (Nikon Instrument Group, Melville, N.Y.). The wounds were measured at different times using the program Image-Pro Plus 4.5.1.29 (Media Cybernetics, LP, Silver Spring, Md.).

Only one member of each inhibitor family was taken as a reference to carry out these experiments in concentrations that do not show any effect on cell proliferation. Thus, the inhibitors used and the concentrations are the following: Troglitazone 5 μM; Ibuprofen 10 μM; Zileuton 10 uM and Zafirlukast 100 nM.

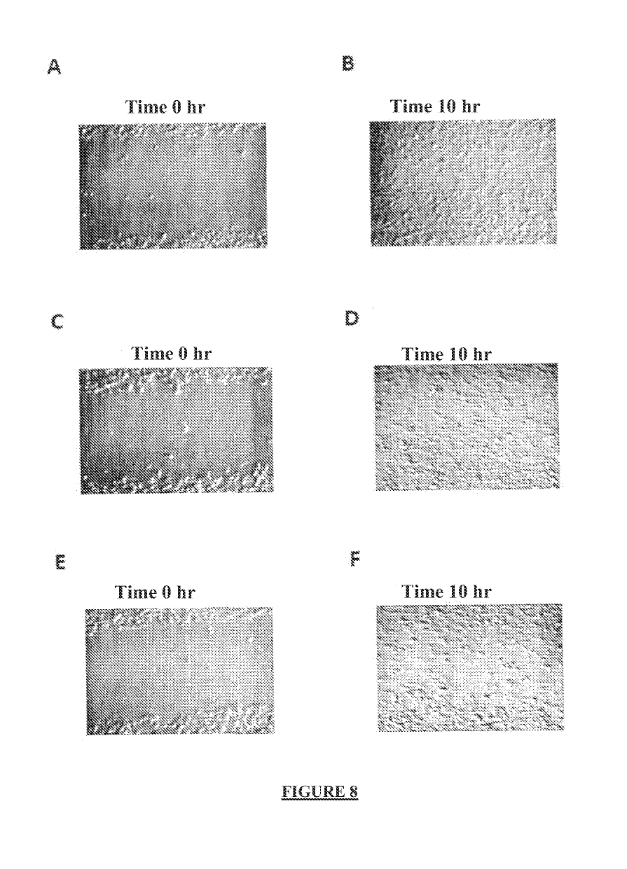

In FIG. 8 are shown the microphotographs A, C and E taken at hour 0 (beginning of the wound) and microphotographs B, D and F taken at the 10th hour from production of the wound, in examples A and B, the culture was carried out in the absence of inhibitors (control). In examples C and D, the culture was carried out in the presence of the Troglitazone and Ibuprofen combination of inhibitors in per se noninhibiting doses. In examples E and F, the culture was carried out in the presence of the combination of three inhibitors, Troglitazone, Ibuprofen and Zileuton, in per se noninhibiting doses.

In the experiment in which the cells were incubated in the absence of inhibitors, the wound totally closes after 10 hours from production of the wound (Figure B). Moreover, the effects of closing are already significant after 4 hours from effecting the wound. And even the double combination of Troglitazone and Ibuprofen using concentrations that do not produce any effect per se on cell proliferation was not capable of inhibit the closing of the wound (Figure D). However, when a combination of three inhibitors in accordance with the invention was used, a significant inhibition of the closing of the wound was observed (Figure F).

In Table 1 below are summarized the results of other experiments on the closing of wounds, carried out as described above, for different inhibitor combinations, always using the concentrations that do not produce an inhibiting effect per se on cell proliferation, indicated in the previous examples (Examples 2 and 4).

TABLE 1

| | % of closing of | |
|---|---|---|
| Treatment | wound (X ± ES) | Significance |
| Control | 100 | |
| Troglitazone/Ibuprofen | 97.3 ± 3.7 | NS |
| Troglitazone/Etoricoxib | 98.1 ± 4.2 | NS |
| Troglitazone/Zileuton | 98.4 ± 3.1 | NS |
| Troglitazone/Zafirlukast | 97.8 ± 4.5 | NS |
| Ibuprofen/Zileuton | 98.3 ± 3.9 | NS |
| Ibuprofen/Zafirlukast | 99.0 ± 3.1 | NS |
| Trog/Zil//Ibu | 73.4 ± 2.9 | p#0.05 |
| Trog/Zaf/Ibu | 75.3 ± 3.8 | p#0.05 |
| Trog/Zil/Eto | 70.4 ± 3.8 | p#0.05 |
| Trog/Zaf/Eto | 72.3 ± 2.9 | p#0.05 |

As showed in Table 1, no double combination using concentrations that do not produce an effect per se on cell proliferation was capable of inhibiting the closing of wounds. However, when the combinations of three inhibitors in accordance with the invention were used, in which one member of the family of acyl-CoA-synthetase inhibitors was always included, a significant inhibition of the closing of wounds was observed. These results coincide with those observed for cell proliferation, and surprising they demonstrate that the combinations of three inhibitor families, with the presence of an acyl-CoA-synthetase inhibitor, are capable, not only of inhibiting cell proliferation, but also of exerting an effect on the aggressive phenotype of these cell lines. It must be emphasized that the closing of wounds takes place through the migratory effect of cells, and not on account of cell proliferation, since 10 hours are not sufficient to observe an effect on cell proliferation.

1. A pharmaceutical composition comprising: i) an acyl-CoA-synthetase enzyme inhibitor (AcsI4), ii) a compound having an inhibiting effect on enzymes with cyclooxygenase activity (COX), and iii) a compound selected from a 5-lipoxygenase enzyme (LOX-5) inhibitor and a leukotriene receptor antagonist.

2. (canceled)

3. (canceled)

4. The pharmaceutical composition of claim 1, wherein the AcsI4 inhibitor comprises a thiazolidinedione.

5. The pharmaceutical composition of claim 4, wherein the AcsI4 inhibitor is selected from Troglitazone and Tosiglitazone, salts, hydrates and solvates thereof.

6. The pharmaceutical composition of claim 1, wherein the COX inhibitor comprises a nonsteroid anti-inflammatory drug (NSAID).

7. The pharmaceutical composition of claim 5, wherein the COX inhibitor is selected from Ibuprofen and Etoricoxib.

8. The pharmaceutical composition of claim 1, wherein the LOX-5 inhibitor is selected from Docebenone (AA-861) and Zileuton.

9. The pharmaceutical composition of claim 1, wherein the leukotriene receptor antagonist compound comprises Zafirlukast.

10. The pharmaceutical composition of claim 1, wherein the composition comprises a combination of i) a thiazolidinedione derivative selected from Troglitazone and Rosiglitazone, ii) Ibuprofen and iii) Docebenone AA-861).

11. The pharmaceutical composition of claim 10, wherein the Troglitazone or Rosiglitazone is present in an amount of up to 15 times less than the therapeutically effective amount to inhibit cell proliferation.

12. The pharmaceutical composition of claim 10, wherein the Ibuprofen is present in an amount of up to 50 times less than the therapeutically effective amount to inhibit cell proliferation.

13. The pharmaceutical composition of claim 10, wherein the Docebenone (AA-861) is present in an amount of up to 50% less than the therapeutically effective amount to inhibit cell proliferation.

14. The pharmaceutical composition of claim 1, wherein the composition comprises a combination of i) a thiazolidinedione derivative selected from Troglitazone and Rosiglitazone, ii) Ibuprofen and iii) Zafirlukast.

15. The pharmaceutical composition of claim 14, wherein the Troglitazone or Rosiglitazone is present in an amount of up to 15 times less than the therapeutically effective amount to inhibit cell proliferation.

16. The pharmaceutical composition of claim 14, wherein the Ibuprofen is present in an amount of up to 50 times less than the therapeutically effective amount to inhibit cell proliferation.

17. The pharmaceutical composition of claim 1, wherein the composition comprises a combination of i) a thiazolidinedione derivative selected from Troglitazone and Rosiglitazone, ii) Ibuprofen and iii) Zileuton.

18. The pharmaceutical composition of claim 17, wherein the Troglitazone or Rosiglitazone is present in an amount of up to 15 times less than the therapeutically effective amount to inhibit cell proliferation.

19. The pharmaceutical composition of claim 17, wherein the Ibuprofen is present in an amount of up to 50 times less than the therapeutically effective amount to inhibit cell proliferation.

20. The pharmaceutical composition of claim 17, wherein the Zileuton is present in an amount of up to 50 times less than the therapeutically effective amount to inhibit cell proliferation.

21. The pharmaceutical composition of claim 1, wherein the composition comprises a combination of i) a thiazolidinedione derivative selected from Troglitazone and Rosiglitazone, ii) Etoricoxib and iii) Zileuton.

22. The pharmaceutical composition of claim 21, wherein the Troglitazone or Rosiglitazone is present in an amount of up to 15 times less than the therapeutically effective amount to inhibit cell proliferation.

23. The pharmaceutical composition of claim 21, wherein the Etoricoxib is present in an amount of up to 100 times less than the therapeutically effective amount to inhibit cell proliferation.

24. The pharmaceutical composition of claim 21, wherein the Zileuton is present in an amount of up to 50 times less than the therapeutically effective amount to inhibit cell proliferation.

25. The pharmaceutical composition of claim 1, wherein the composition comprises a combination of i) a thiazolidinedione derivative selected from Troglitazone and Rosiglitazone, Etoricoxib and iii) Zafirlukast.

26. The pharmaceutical composition of claim 25, wherein the Troglitazone or Rosiglitazone is present in an amount of up to 15 times less than the therapeutically effective amount to inhibit cell proliferation.

27. The pharmaceutical composition of claim 25, wherein the Etoricoxib is present in an amount of up to 100 times less than the therapeutically effective amount to inhibit cell proliferation.

28. A method of inhibiting cancer cell proliferation in a patient afflicted with cancer cell proliferation, the method comprising administering to a patient in need thereof a combination of: i) an acyl-CoA-synthetase enzyme inhibitor (AcsI4), ii) a compound having an inhibiting effect on enzymes with cyclooxygenase activity (COX), and iii) a compound selected from a 5-lipoxygenase enzyme (LOX-5) inhibitor and a leukotriene receptor antagonist: in an amount and for a period of time effective to inhibit the cancer cell proliferation.

29. The method of claim 28, wherein the cancer is breast cancer or colon cancer.

30. A method of treating a patient afflicted with psoriasis, the method comprising administering to a patient in need thereof a combination of: i) an acyl-CoA-synthetase enzyme inhibitor (AcsI4), ii) a compound having an inhibiting effect on enzymes with cyclooxygenase activity (COX), and iii) a compound selected from a 5-lipoxygenase enzyme (LOX-5) inhibitor and a leukotriene receptor antagonist; in an amount and for a period of time effective to treat the psoriasis.
